(19) United States
(12) Patent Application Publication
(10) Pub. No.: US 2025/0302009 A1
(43) Pub. Date: Oct. 2, 2025

(54) SYSTEM AND METHOD FOR DETECTING ANIMAL LAMENESS & CHARACTERIZING ANIMAL HEALTH

(71) Applicant: Companion Professional, Corte Madera, CA (US)

(72) Inventors: Oliver Dain, Corte Madera, CA (US); Paul Mundell, Corte Madera, CA (US); John Honchariw, Corte Madera, CA (US); Libby Rockaway, Corte Madera, CA (US)

(21) Appl. No.: 19/094,459

(22) Filed: Mar. 28, 2025

Related U.S. Application Data
(60) Provisional application No. 63/572,021, filed on Mar. 29, 2024.

(51) Int. Cl. A01K 29/00; A61B 5/00; A61B 5/11; G06V 10/62; G06V 10/77; G06V 10/98; G06V 40/10; G06V 40/20; G16H 15/00; G16H 40/20; G16H 50/30; G16H 80/00

(52) U.S. Cl. CPC A01K 29/005; A61B 5/1116; A61B 5/1128; A61B 5/7275; G06V 10/62; G06V 10/7715; G06V 10/987; G06V 40/10; G06V 40/20; G16H 15/00; G16H 40/20; G16H 50/30; G16H 80/00; A61B 2503/40; A61B 2505/07

(57) ABSTRACT

One variation of a method includes: receiving a video of an animal executing a series of target movements, the video captured by a device accessed by an owner of the animal; from the video, extracting a set of body data representing movement of a set of body features of the animal; accessing a lameness model linking body data extracted from videos of animals to lameness of a set of lameness types in animals; based on the set of body data and the lameness model, predicting lameness of a first lameness type exhibited by the animal in a first segment of the video; in response to detecting lameness of the first lameness type for the animal, generating a report indicating detection of lameness of the first lameness type and including the first segment of the video; and transmitting the report to an animal health professional, affiliated with the animal, for review.

### CROSS-REFERENCE TO RELATED APPLICATIONS

This application claims the benefit of U.S. Provisional Application No. 63/572,021, filed on 29 Mar. 2024, which is incorporated in its entirety by this reference.

This application is also related to U.S. patent application Ser. No. 17/886,373, filed on 11 Aug. 2022, and U.S. patent application Ser. No. 17/886,378, filed on 11 Aug. 2022, each of which is incorporated in its entirety by this reference.

### TECHNICAL FIELD

This invention relates generally to the field of animal health and, more specifically, to a new and useful system and method for detecting animal lameness and characterizing animal health in the field of animal health.

### BRIEF DESCRIPTION OF THE FIGURES

FIG. 1 is a flowchart representation of a method;

FIG. 2 is a flowchart representation of one variation of the method;

FIG. 3 is a flowchart representation of one variation of the method;

FIG. 4 is a flowchart representation of one variation of the method; and

FIG. 5 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

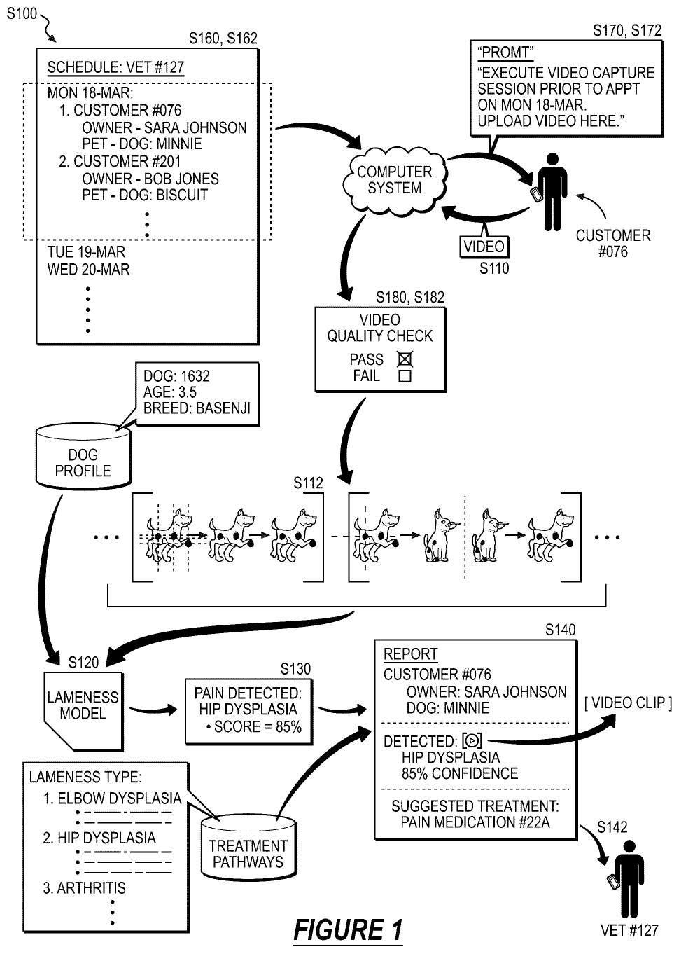

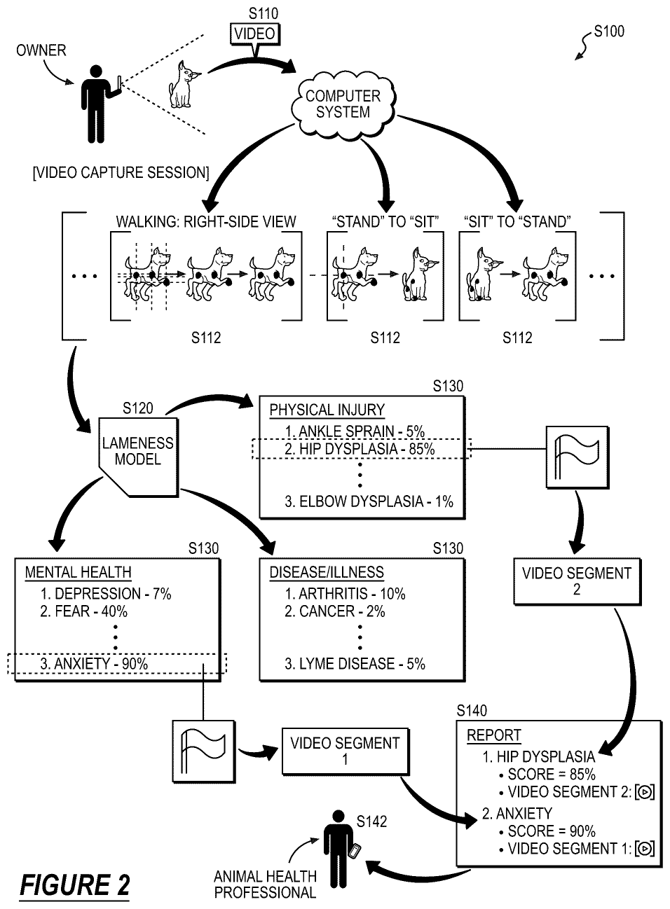

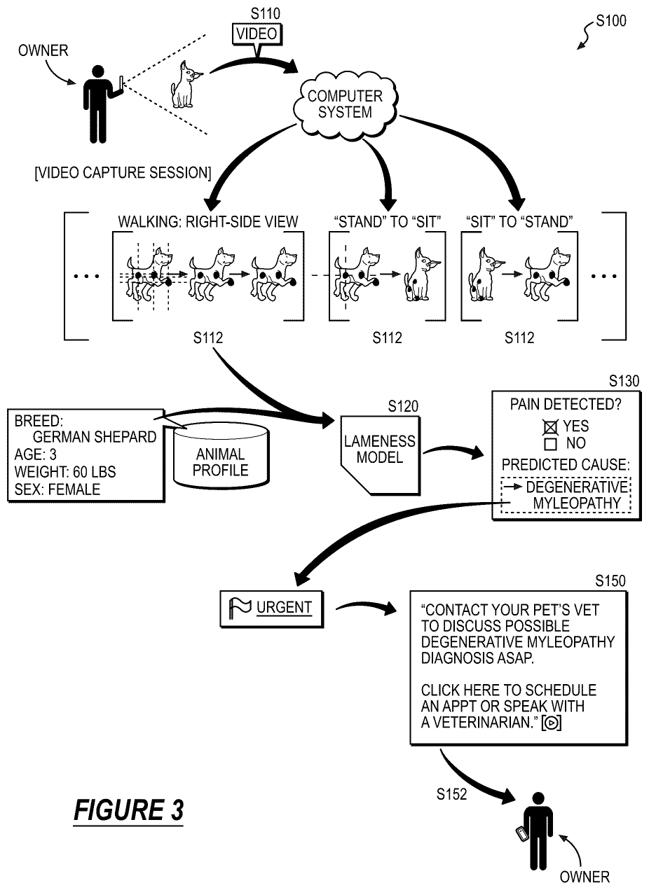

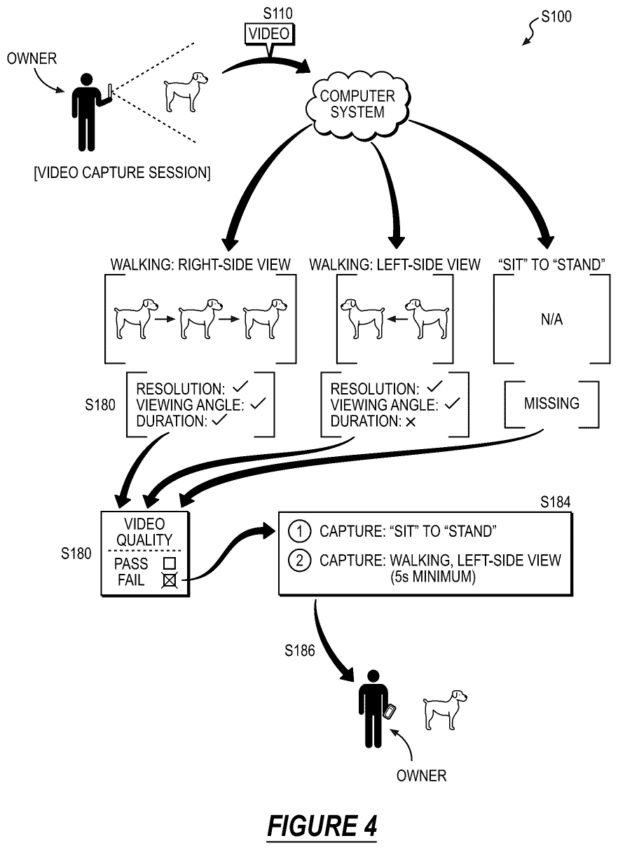

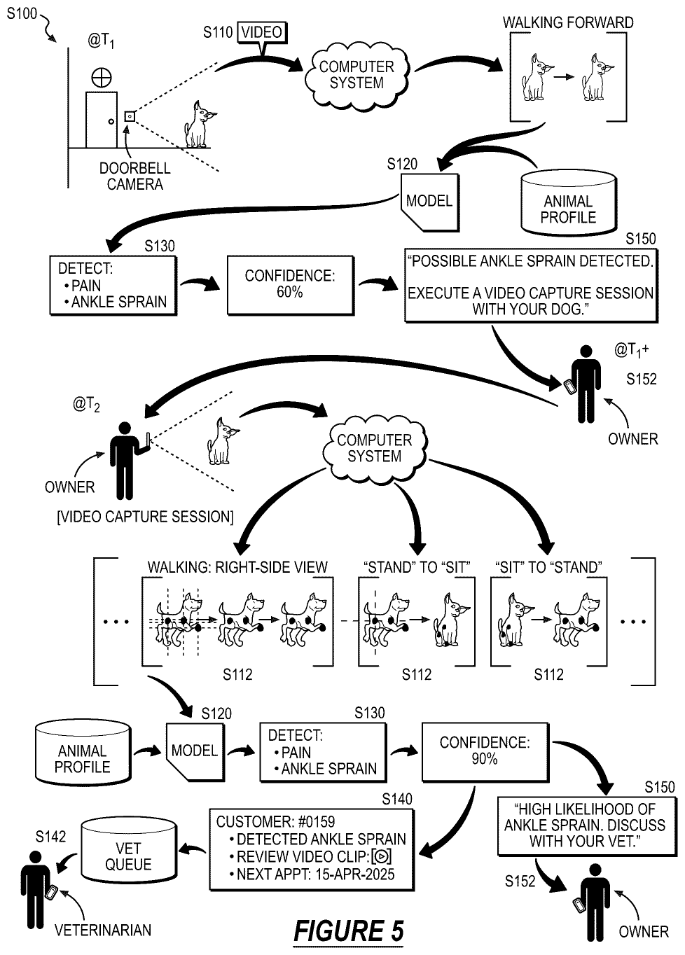

As shown in FIGS. 1-5, a method S100 includes: receiving a video of an animal executing a series of target movements during a first video capture session, the video captured by a first computing device accessed by an owner of the animal in Block S110; extracting a set of body data from the video, the set of body data representing movement of a set of body features of the animal depicted in the video in Block S112; accessing a lameness model linking body data extracted from videos of animals to lameness of a set of lameness types in animals in Block S120; and, based on the set of body data and the lameness model, predicting lameness of a first lameness type exhibited by the animal in a first segment of the video in Block S130. The method S100 further includes, in response to detecting lameness of the first lameness type for the animal: generating a report indicating detection of lameness of the first lameness type in Block S140; populating the report with the segment of the video corresponding to lameness of the first lameness type in Block S140; and transmitting the report to a second computing device accessed by an animal health professional affiliated with the animal in Block S142.

In one variation, in response to detecting lameness of the first lameness type for the animal, the method S100 further includes: generating a second report indicating detection of lameness of the first lameness type and including a prompt to review the second report with the animal health professional in Block S150; and transmitting the second report to the owner at the first computing device in Block S152.

In one variation, in response to receiving the video from the first computing device, the method S100 further includes: characterizing a quality of the video in Block S180; in response to the quality exceeding a threshold quality, approving the video for analysis in Block S182; and, in response to approving the video for analysis, extracting the set of body data from the video in Block S112. In this variation, the method S100 further includes, in response to the quality falling below the threshold quality: generating a first owner prompt to record a second video of the animal executing a subseries of target movements, in the series of target movements, during a second video capture session in Block S184; and transmitting the first owner prompt to the owner at the first computing device in Block S186.

One variation of the method S100 includes: receiving a video of an animal executing a series of target movements during a first video capture session in Block S110; extracting a set of body data from the video, the set of body data representing characteristics of a set of body features of the animal depicted in the video in Block S112; accessing a lameness model linking body data extracted from videos of animals to lameness of a set of lameness types in animals in Block S120; and, based on the set of body data and the lameness model, detecting lameness of a first lameness type exhibited by the animal in a first segment of the video in Block S130. In this variation, in response to detecting lameness of the first lameness type for the animal, the method S100 further includes: generating a report indicating detection of lameness of the first lameness type in Block S140; populating the report with the segment of the video corresponding to lameness of the first lameness type in Block S140; and transmitting the report to a first computing device accessed by a user affiliated with the animal in Block S142.

### 1.1 Method: Appointment Schedule

As shown in FIG. 1, one variation of the method S100 includes: at a first time, accessing a schedule defined for an animal health professional in Block S160; and identifying a first appointment for the animal and specified by the schedule, the first appointment scheduled at a second time succeeding the first time and falling within a threshold duration of the first time in Block S162. The method S100 further includes, in response to identifying the first appointment: generating a first owner prompt to capture a video of the animal executing a series of target movements during a video capture session in Block S170; and transmitting the first owner prompt to an owner of the animal via an instance of an owner portal executing on a first computing device accessed by the owner in Block S172. The method S100 further includes, in response to receiving the video of the animal the instance of the owner portal in Block S110: extracting a set of body data from the video, the set of body data representing movement of a set of body features of the animal depicted in the video in Block S112; accessing a lameness model linking body data extracted from videos of animals to lameness of a set of lameness types in animals in Block S120; and, based on the set of body data and the lameness model, detecting lameness of a first lameness type exhibited by the animal in a first segment of the video in Block S130.

The method S100 further includes, in response to detecting lameness of the first lameness type for the animal: generating a report indicating detection of lameness of the first lameness type in Block S140; extracting a portion of the video depicting lameness of the first lameness type exhibited by the animal; populating the report with a prompt to review the portion of the video; and, at a third time succeeding the first time and preceding the second time, transmitting the report to an animal health professional for review via an instance of a veterinarian portal associated with the animal health professional in Block S140.

One variation of the method S100 includes: at a first time, accessing a schedule defined for a veterinarian; identifying an appointment scheduled by a user for a dog—affiliated with the user—at a second time falling within a target duration of the first time; generating a first owner prompt to capture a video of the dog executing a session protocol during a video capture session and transmitting the first owner prompt to the user via an instance of a user portal executing on a first computing device (e.g., a smartphone, a tablet) accessed by the user; in response to receiving the video—captured by a camera integrated into the first computing device—of the dog, characterizing a quality of the video; and, in response to the quality of the video exceeding a threshold quality, approving the video for analysis. Then, in response to approving the video, the method S100 further includes: extracting a set of body data—representing position and/or movement of the dog and/or of a set of body features (e.g., head, feet, knees, hips)—from the video in Block S112; accessing a lameness model linking body data extracted from videos of dogs to lameness of a set of lameness types in dogs in Block S120; and characterizing lameness exhibited by the dog in the video based on the set of body data and the lameness model in Block S130. The method S100 further includes, in response to detecting an instance of lameness of a first lameness type for the dog: generating a report indicating detection of the instance of lameness of the first lameness type in Block S140; populating the report with a prompt to review detection of the instance of lameness and/or a portion of the video associated with detection of the instance of lameness; and, at a third time succeeding the first time and preceding the second time, transmitting the report to the veterinarian for review prior to the appointment with the dog in Block S142.

### 2. Applications

Generally, the method S100 can be executed by a computer system (e.g., a computer network, a remote computer system, a local or remote server) and/or by an application (e.g., a native application, a web application) to: access a video of a dog—executing a series of movements, such as walking back and forth, transitioning from a “sit” position to a “stand” position, etc.—captured on the dog owner's mobile device during a video capture session; detect instances of lameness—which may affect the dog's health and/or pain experienced by the dog—based on characteristics of the dog and/or the dog's movements extracted from the video; and selectively notify the dog's owner and/or a veterinarian associated with the dog of detected instances of lameness, such as for further investigation and/or for implementation of a particular treatment pathway corresponding to a type of lameness detected for the dog.

In particular, the computer system can periodically prompt a user (i.e., the dog's owner)—such as via the application executing on a mobile device accessed by the user—to execute a video capture session with her dog according to a video session protocol. For example, the computer system can prompt the user to capture video of the dog: from a rear-facing view with the dog walking directly away from a camera integrated into the user's mobile device; from a frontward-facing view with the dog walking directly toward the camera; from a first side-facing view with the dog walking and oriented approximately 90-degrees from the camera; from a second-side facing view—opposite the first side-facing view—with the dog walking and oriented approximately 90-degrees from the camera; from the frontward-facing view with the dog transitioning from a “sit” position to a “stand” position; from the rear-facing view with the dog transitioning from the “sit” position to the “stand” position; from the first and/or second side-facing view with the dog transitioning from the “sit” position to the “stand” position; etc. The computer system can then implement a lameness model—linking characteristics of dog movements, postures, and/or expressions (e.g., facial expressions) to lameness in dogs—to: extract a set of body data—representing position and/or movement of the dog and/or of particular body features (e.g., head, feet, knees, hips) of the dog—from the video; and detect instances of lameness for the dog based on the set of body data extracted from the video.

Furthermore, the computer system can predict a particular type of lameness, from a corpus of lameness types, exhibited by the dog in the video, such as corresponding to a sprain, a fracture, dysplasia (e.g., hip or elbow dysplasia), arthritis, cancer, Lyme disease, broken or overgrown toenails, etc. For example, the computer system can leverage the lameness model to predict a lameness score—such as represented by a score between one and five, between 0% and 100%, etc.—for each lameness type in the set of lameness types. In particular, in this example, the computer system can derive: a first lameness score of 0% for an ankle sprain; a second lameness score of 80% for hip dysplasia; etc. In this example, the computer system can then: generate a report indicating the second lameness score of 80% for hip dysplasia and including a prompt to review the report—and/or a segment of the video corresponding to detection of hip dysplasia for the dog—with the dog's veterinarian; and transmit this report to the user and/or directly to the dog's veterinarian for further investigation.

The computer system can, therefore, detect instances of lameness that may otherwise go undetected by the user, thereby enabling the user to seek professional health care for her dog and/or implement a corresponding treatment pathway earlier, such as prior to worsening of a particular condition exhibited by the dog and/or with sufficient time to implement the corresponding treatment pathway.

In one implementation, the computer system can interface with both: an owner portal executing on a mobile device accessed by a user (i.e., the dog owner) associated with the dog; and a veterinarian portal executing on a computing device (e.g., a smartphone, a tablet, a laptop, a desktop computer) accessed by the dog's veterinarian or any other animal health professional. In this implementation, the computer system can automatically generate and transmit reports—summarizing insights derived from videos captured during video capture sessions with the dog and related to dog health—to the veterinarian via the veterinarian portal for review, such as prior to an upcoming appointment for the dog with the veterinarian. For example, the computer system can: access an appointment schedule defined for the veterinarian; identify an appointment for the dog scheduled at a time within the next week; generate an owner prompt to upload a video of her dog—executing a capture session protocol during a video capture session accordingly—via the application; transmit the owner prompt to the user via the owner portal; scan the video and implement a lameness model to detect instances of lameness exhibited by the dog in the video; generate a report detailing any instances of lameness detected in the video and/or any other key information extracted from the video; generate a veterinarian prompt to review the report and/or the video prior to the appointment with the dog and the dog's owner; and transmit the veterinarian prompt to the veterinarian for review of the report and/or the video prior to the appointment.

The computer system can therefore: enable early and/or rapid detection of any instances of lameness detected in the video; enable the veterinarian to review detected instances of lameness prior to the appointment and, therefore, focus on treating instances of lameness and/or discussing corresponding information with the dog owner during the appointment; and thus minimize risk of missing detection of lameness—detected in the video—during the appointment with the veterinarian.

Furthermore, in one variation, the computer system can access ambient video recorded by one or more optical sensors integrated into home devices—such as a video doorbell and/or a home security system—installed in the user and/or dog's home. In this variation, the computer system can leverage these ambient videos—such as in combination with videos captured during scheduled and/or periodic video capture sessions—to enable earlier and more accurate detection of lameness and/or any other health-related issues exhibited by the dog. In particular, in one example, the computer system can selectively prompt the user to execute a video capture session—such as according to a defined video session protocol—with the dog in response to predicting an instance of lameness for the dog based on body data extracted from an ambient video captured by a video doorbell installed at the dog's home. Therefore, the computer system can: predict an instance of lameness for the animal—based on the ambient video captured by the video doorbell—at a first confidence level; in response to predicting the instance of lameness, prompt the user to execute a video capture session with her dog; and then confirm and/or reject this predicted instance of lameness—at a second confidence level exceeding the first confidence level—based on body data extracted for the dog in a video captured during the video capture session. Therefore, the computer system can leverage lower-resolution ambient video—captured by ambient sensors installed throughout the dog's home—to initially predict instances of lameness for the dog and then confirm and/or reject these predictions based on higher-resolution video captured during video capture sessions between the user and the dog.

The computer system is described below as executing Blocks of the method S100 to detect instances of lameness (e.g., characterized by pain, injury) for a dog based on poses, movements, etc., of the dog during video capture sessions. However, the computer system can execute these Blocks of the method in order to detect instances of lameness for any other type of animal, such as a cat, a horse, or a bird.

Furthermore, the computer system is described below as executing Blocks of the method S100 to notify a veterinarian—affiliated with an animal—of detection of lameness of a particular lameness type for the animal. However, the computer system can execute these Blocks of the method in order to notify any other type of animal health professional—such as a veterinary technician or technologist, an animal nutritionist, an animal physiotherapist, an animal behaviorist or trainer, etc.—of detection of lameness of a particular lameness type for the animal.

### 3. Application+Animal Onboarding

Generally, the computer system can interface with a native application or web application executing on a computing device accessed by a user (e.g., a pet owner) affiliated with a dog. In one implementation, the computer system can prompt the user to generate a dog profile for her dog within the native application. For example, the user may download a native application to her smartphone or navigate to a web application within a browser executing on her smartphone. The computer system can then: generate a prompt to create a dog profile for her dog within the application and manually populate the dog profile with various information, such as a name, breed, age, size (e.g., weight, height, length), and/or primary coat colors of her dog; and transmit the prompt to the user via the application. The computer system can then: receive this information from the user via the application; and store the dog profile—populated with the dog's information—in a remote database.

Additionally or alternatively, in another example, the computer system can automatically populate the dog profile with dog characteristics extracted from an image or video of the dog recorded by the user. For example, in response to the user downloading the native application to her smartphone, the computer system can: generate a prompt to capture a video of the dog via a camera integrated in the user's smartphone; transmit the prompt to the user; in response to receiving the video, derive a set of dog characteristics—such as including a breed, a size, a set of primary coat colors of the dog's coat, etc.—of the dog based on features extracted from frames of the video; and populate a dog profile—generated for the dog—with the set of dog characteristics.

In one implementation, the computer system can: transmit prompts to the user and receive videos from the user via an instance of an owner portal—executing on a computing device accessed by the user (e.g., within the application)—associated with the user; and/or transmit prompts, reports, and/or videos of the animal to an animal health professional (e.g., a veterinarian), affiliated with the animal, via an instance of a veterinarian portal—executing on a computing device accessed by the animal health professional—associated with the animal health professional. In this implementation, the computer system can thus enable communication and/or sharing of data (e.g., video) between the owner and veterinarian portals.

Additionally and/or alternatively, in one implementation, the computer system can interface with a patient management system (e.g., a cloud platform) employed by an animal health professional and/or animal health network. In this implementation, the computer system can both transmit prompts, reports, and/or videos of the animal to an animal health professional, and receive requests for video and/or other communications from the animal health professional via the patient management system. The computer system can therefore enable the animal health professional to access this information (e.g., reports, videos)—and/or request information—regarding an animal directly within the patient management system already implemented by the animal health professional, rather than requiring the animal health professional to access an additional external tool or application.

### 3.1 Animal Model

Generally, the computer system can implement an animal model to detect presence, position, and/or movement of a dog in images and/or videos of the dog. In one implementation, the computer system can access a particular animal model—such as a dog presence, movement, transition, and/or pose detection model—trained on images of dogs of an age, breed, size, shape, and/or coat length, etc. that are the same or similar to characteristics stored in the dog's profile. Similarly, the computer system can: tune a generic animal model based on various characteristics stored in the dog profile; or select one animal model—from a corpus of existing animal models—developed to detect presence and/or pose of dogs exhibiting various characteristics similar to those of the dog. Alternatively, the computer system can implement a generic animal model to detect presence, pose, position, orientation, etc., of the dog within the field of view, such as if limited information about the dog is provided by the user during setup. The computer system can then implement this animal model to detect presence (i.e., location and orientation) and pose of the dog in video or images recorded by a camera integrated into the user's mobile device (e.g., a smartphone, a tablet). By accessing an animal model “tuned” to detect presence and pose of animals exhibiting characteristics similar to those aggregated into the dog's profile during setup, the computer system can detect presence, position, and/or orientation of the dog in a video or image more quickly and with increased confidence.

### 4. Video Capture Session

Block S170 of the method S100 recites: generating a first owner prompt to capture a video of the animal executing a series of target movements during a video capture session. Furthermore, Block S172 of the method S100 recites transmitting the first owner prompt to an owner of the animal.

Generally, once the computer system has accessed the foregoing data, the computer system can prompt the user (i.e., the dog owner) to initiate a video capture session for the dog. In particular, the computer system can: generate a prompt to locate the dog within a particular space—co-occupied by the user—in preparation for a video capture session; and transmit the prompt to the user (e.g., via push notification, via text message). For example, the computer system can transmit the prompt to the user via an instance of an owner portal executing on a computing device (e.g., a smartphone, a tablet, a desktop computer) accessed by the user.

Then, in response to receiving confirmation from the user that the dog is located in the particular space (e.g., with the user) and that the user is ready to begin a video capture session, the computer system can generate one or more prompts to: locate the dog within a field of view of a camera integrated into the user's mobile device; initiate a video recording of the dog within the field of view at a start of the video capture session; and promote (e.g., via voice command) execution of a series of movements by the dog—within the field of view of the camera—during the video capture session. The computer system can thus transmit these one or more prompts to the user to guide execution of the video capture session. In response to completion of the video capture session, the computer system can: access the video recording captured by the user during the video capture session; and implement the animal model to detect the dog and/or track motion and postures of the dog in the video recording.

In one implementation, the computer system can prompt the user to capture a video recording of the dog executing a series of poses and/or movements according to a video session protocol configured to highlight pain, injury, illness, etc. experienced by the dog. For example, prior to a video capture session, the computer system can load a video session protocol locally onto the application for execution with the dog and/or the user during the video capture session. In this example, the computer system can select a video session protocol configured to enable evaluation of the dog's postures and/or movements during a set of transition poses (e.g., “stand to sit” transition pose, “sit to down” transition pose, “down to stand” transition pose) and/or and the dog's gait (e.g., posture, velocity, stride length, balance, weight distribution, duration) while walking.

In particular, in one example, the computer system can prompt the user to capture video of the dog: from a rear-facing view with the dog walking directly away from the camera in a first direction; from a frontward-facing view with the dog walking directly toward the camera in a second direction opposite the first direction; in a first side-facing view with the dog walking in a third direction and oriented approximately 90-degrees from the camera; in a second-side facing view with the dog walking a fourth direction—opposite the third direction—and oriented approximately 90-degrees from the camera; etc. The computer system can also prompt the user to capture video of the dog: from the frontward-facing view with the dog transitioning from a “sit” position to a “stand” position; from the first and/or second side-facing view with the dog transitioning from the “sit” position to the “stand” position; from the frontward-facing view with the dog transitioning from the “stand” position to a “lie-down” position; from the first and/or second side-facing view with the dog transitioning from the “stand” position to the “lie-down” position; etc.

In one variation, the computer system can: automatically update the video session protocol in real-time based on characteristics of the dog and/or data derived from the video; and prompt the user (e.g., dog owner) to execute the updated session protocol accordingly. For example, during capture of the video during a video capture session, in response to detecting failure of the dog to complete a particular movement, in the sequence of movements defined by the video session protocol, the computer system can automatically modify the video session protocol to exclude the particular movement and/or include a new movement in replacement of the particular movement. The computer system can then prompt the user to execute this new movement during the video capture session in real-time.

In one variation, the computer system can provide an instructional video to the user—outlining a video session protocol for executing with her animal during a video capture session—prior to initiation of the video capture session with her animal. For example, at a first time, the computer system can: generate a first prompt to locate the dog within a particular space—co-occupied by the user—in preparation for a video capture session; and transmit the prompt to the user (e.g., via push notification, via text message). Then, in response to receiving confirmation from the user that the dog is located in the particular space with the user, the computer system can: generate a second prompt—including an instructional video linked to the second prompt—to review the instructional video prior to initiating the video capture session; and transmit the second prompt to the user.

In one example, the computer system can access an instructional video depicting an animal and/or an instructor (e.g., an owner of the dog, an animal trainer) executing the session protocol, including executing a series of movements such as walking in a series of directions and/or orientations relative a camera recording the instructional video and/or transitioning between a series of poses (e.g., “sit,” “stand,” “lie-down”). In response to completion of playback of the instructional video, the computer system can: generate a third prompt to: locate the dog within a field of view of a camera integrated into the user's mobile device; initiate a video recording of the dog within the field of view at a start of the video capture session; and promote (e.g., via voice command) execution of the session protocol by the dog during the video capture session, as described above.

Additionally and/or alternatively, in one variation, the computer system can provide an audio guide configured to playback during capturing of the video, such that the user (e.g., dog owner) may listen to the audio guide while executing the video capture session with the animal.

### 4.1 Video Recording+User Feedback

Block S110 of the method S100 recites receiving a video of an animal executing a series of target movements during a first video capture session, the video captured by a first computing device accessed by an owner of the animal.

Generally, the computer system can receive a video recording—captured by a computing device (e.g., a smartphone, a tablet) accessed by the dog owner—of the dog executing a series of poses and/or movements according to a video session protocol configured to highlight pain, injury, illness, etc. experienced by the dog, as described above. For example, the computer system can receive the video of the animal—executing the series of target movements—via an instance of an owner portal executing on the owner's mobile device. In particular, in one example, the computer system can receive the video of the animal executing the series of target movements—such as including walking in a first direction along a pathway within a field of view of an optical sensor integrated within the computing device of the owner, walking in a second direction opposite the first direction and along the pathway, transitioning from a “sit” position to a “stand” position, etc.—via the instance of the owner portal.

### 4.1.1 User Feedback+Video Quality

In one variation, Block S180 of the method S100 recites, in response to receiving the video from the first computing device, characterizing a quality of the video in Block S180. Furthermore, Block S182 of the method S100 recites, in response to the quality exceeding a threshold quality, approving the video for analysis. Alternatively, Blocks S184 and S186 of the method S100 recite, in response to the quality falling below the threshold quality: generating a first owner prompt to record a second video of the animal executing a subseries of target movements, in the series of target movements, during a second video capture session; and transmitting the first owner prompt to the owner at the first computing device.

Generally, in this variation, the computer system can provide feedback to the user regarding a quality of a video captured by the user during the video capture session.

In particular, the computer system can derive insights with higher resolution and/or increased accuracy from videos of relatively higher quality. Therefore, in one implementation, if the computer system receives a video of quality less than a threshold quality, the computer system can prompt the user to record a new video—in replacement and/or in addition to the original video—in order to improve accuracy of insights derived from video captured during a video capture session. For example, the computer system can characterize a quality of the video as “low” quality in response to the video: exhibiting a relatively low frame rate and/or pixel resolution; depicting the dog at a distance exceeding a maximum distance from the camera, such that the dog is too far away from the camera; depicting the dog at a distance falling below a minimum distance from the camera, such that the dog is too close to the camera; omitting one or more movements or transitions—such as a transition from a “sit” pose to a “stand” pose—defined by the session protocol; etc.

In one example, in response to characterizing the quality of the video as “low” quality, the computer system can: generate a notification indicating characterization of the video as “low” quality and including a rationale—such as a low frame rate, a low resolution, a low quality view of the dog, etc.—for characterizing the video as “low” quality; append the notification with a prompt to capture a new video of the dog executing the session protocol and/or executing a portion of the session protocol; and transmit the notification to the user via the application. Then, in response to receiving a new video of the dog from the user, the computer system can repeat this process to characterize quality of the new video and provide feedback to the user accordingly. In particular, in this example, in response to characterizing the new video as “high” quality, the computer system can: generate a notification indicating approval of the “high” quality video; and transmit the notification to the user.

The computer system can thus automatically accept or reject a video—recorded during the video capture session—based on a derived quality of the video. The computer system can then provide post-hoc feedback to the user regarding the quality of the video in order to ensure sufficient quality of videos provided for this animal and therefore enable detection of animal health—such as characterized by lameness and/or body condition—with increased accuracy.

Additionally or alternatively, in another implementation, the computer system can provide real-time feedback to the user—during recording of the video within the video capture session—in order to promote capturing of high-quality video of the dog. In one example, the computer system can: render a rectangular outline on a display of the mobile device during recording of the video within the video capture session; and prompt the user to locate and/or “fit” the dog within the rectangular outline, such that the dog remains within a target region of the field of view and/or at a target distance from the camera. Furthermore, the computer system can: modify a color of the rectangular outline in order to provide feedback to the user regarding whether the dog is properly located within the field of view, such as by: rendering a red rectangular outline if the dog is located outside the target region defined by the rectangular outline; rendering a yellow rectangular outline if a portion of the dog is located outside the target region defined by the rectangular outline; and/or rendering a green rectangular outline if the dog is located within the target region defined by the rectangular outline. In another example, the computer system can: prompt the user (e.g., in real-time) to move away from the dog in response to detecting the dog at a distance less than a minimum distance from the camera; and/or prompt the user (e.g., in real-time) to move toward the dog in response to detecting the dog at a distance exceeding a maximum distance from the camera.

Therefore, in this implementation, the computer system can provide real-time feedback (e.g., visual and/or graphical feedback) to the user—during the video capture session—to promote capturing of high-quality video and thus: increase quality of video captured by the user and therefore detect injury, pain, etc. experienced by the dog with increased accuracy; minimize instances of low-quality videos captured by the user and therefore reduce a quantity of repeat video capture sessions; and minimize time required by the user capturing video of the dog.

### 5. Lameness Detection

The computer system can characterize lameness for the dog based on features extracted from a video of the dog captured by the user—and uploaded via the application—during a video capture session. For example, the computer system can detect lameness corresponding to: acute injuries, such as including a sprain, a strain, a fracture, a dislocation, etc.; a chronic condition, such as including elbow or hip dysplasia, arthritis, cancer, etc.; and/or an acute condition exhibited by the dog, such as including broken or overgrown nails, an overgrown coat, stress due to an environment occupied by the dog, etc.

Generally, the computer system is described as executing Blocks of the method S100 to detect instances of lameness—such as indicative of animal pain, injury (e.g., fractures, wounds, muscle tears), health issues (e.g., infection, arthritis, obesity, nutritional deficiency), genetic conditions, etc.—for an animal based on postures, movements, body characteristics (e.g., size, weight, facial expressions), etc., of the animal during video capture sessions. However, the computer system can execute Blocks of the method in order to leverage body data—such as associated with position of body features (e.g., joints, limbs) during execution of movements and/or postures, weight, body condition score, dermatological features (e.g., coat color, coat coverage), behaviors (e.g., tail wagging size and frequency, vocalizations), facial expressions, etc.—extracted from video recordings of the animal to detect instances of any health-related condition for the animal, such as including pain, obesity, dermatological issues, neurological issues, anxiety, depression, mood (e.g., happiness, sadness), energy level, disease (e.g., cancer, metabolic disease), etc.

### 5.1 Body Data

Block S112 of the method S100 recites: extracting a set of body data from the video, the set of body data representing movement of a set of body features of the animal depicted in the video.

Generally, the computer system can extract a set of body data—representing position and/or movement of the dog and/or of particular body features (e.g., head, feet, knees, hips) of the dog during the video capture session—from the video captured during the video capture session for the dog. In particular, the computer system can extract a set of body data—such as including a sequence of locations of the dog within a working field, a sequence of relative positions of various body features (e.g., head, feet, knees, hips) of the dog's body, velocities of the dog's body during particular transitions or movements, a weight distribution of the dog in a particular pose and/or during a particular transition or movement, etc.—corresponding to the dog's gait, poses, and/or transitions between poses during the video capture session. The computer system can then leverage this set of body data to detect and/or characterize lameness—which may correspond to various physiological and/or neurological health issues—exhibited by the dog in the video. For example, the computer system can leverage this set of body data to detect abnormalities—such as including altered weight-bearing, asymmetrical stride length, irregular joint flexion, relatively slow transition between poses (e.g., from “sit” to “stand”), relatively slow walking velocity, etc.—associated with animal injury, disease, mental health disorders, and/or pain to detect lameness exhibited by the animal in the video.

In one implementation, the computer system extracts the set of body data—from the video of the animal executing the target sequence of movements—representing: movement of a set of anatomical features (e.g., joints, limbs, head, paws) of the animal during execution of the series of target movements; and facial expressions of the animal during execution of the series of target movements.

### 5.2 Lameness Model

Block S130 of the method S100 recites accessing a lameness model linking body data extracted from videos of animals to lameness of a set of lameness types in animals.

Generally, the computer system can implement a lameness model—linking characteristics of dog movements, postures, and/or expressions (e.g., facial expressions) to lameness in dogs—to detect instances of lameness for an animal in images and/or video of the dog. For example, the computer system can implement machine learning, regression, artificial intelligence, and/or other techniques to train the lameness model. The computer system can then implement this lameness to: extract a set of body data (e.g., as described above)—representing position and/or movement of the dog and/or of particular body features (e.g., head, feet, knees, hips) of the dog—from the video; and detect instances of lameness for the dog based on the set of body data extracted from the video.

In one implementation, the computer system can select a particular lameness model—from a corpus of existing lameness models—trained on images and/or video of dogs of an age, breed, size, etc. corresponding to characteristics of the dog (e.g., stored in the dog profile) and developed to detect lameness in dogs exhibiting characteristics similar to the dog. Similarly, the computer system can tune a generic lameness model based on characteristics stored in the dog profile. Additionally or alternatively, in another implementation, the computer system can select a particular lameness model—from a corpus of existing lameness models—trained on images and/or video of dogs exhibiting lameness of a particular lameness type, such as corresponding to a sprain, a fracture, dysplasia, arthritis, cancer, broken or overgrown toenails, etc. Alternatively, in another implementation, the computer system can implement a generic lameness model developed to detect lameness in dogs based on images or video of dogs. In each of these implementations, the computer system can then implement this lameness model to detect lameness—such as characterized by injury or pain exhibited by the dog—in images or video recorded by a camera integrated into the user's mobile device during a video capture session.

### 5.3 Lameness Type

Block S130 of the method S100 recites, based on the set of body data and the lameness model, predicting lameness of a first lameness type exhibited by the animal in a first segment of the video.

Generally, the computer system can predict presence and/or absence of lameness of a set of lameness types—such as corresponding to physical ailments, mental health disorders, pain, etc.—for the animal based on the lameness model and body data extracted from the video of the animal executing the series of target movements.

In one implementation, the computer system can predict lameness of a first subset of lameness types, in the set of lameness types, corresponding to physical ailments, such as including physical injuries (e.g., an ankle sprain, hip dysplasia) and/or physical diseases (e.g., malnutrition, infection, genetic conditions). For example, the computer system can: receive a video of an animal executing a series of target movements during a video capture session; extract a set of body data from the video; and, based on a first subset of body data in the set of body data—the first subset of body data representing movement of a set of limbs (e.g., paws, forelegs, hindlegs) in the set of body features of the animal—and the lameness model, detect lameness of a lameness type (e.g., a sprain, arthritis, hip dysplasia, a paw injury, a nail injury, degenerative disc disease) corresponding to a physical ailment or injury associated with a particular limb in the set of limbs.

Additionally or alternatively, in another implementation, the computer system can predict lameness of a second subset of lameness types, in the set of lameness types, corresponding to mental health disorders, such as including anxiety and/or depression. For example, the computer system can: receive a video of an animal executing a series of target movements during a video capture session; extract a set of body data from the video; and, based on a second subset of body data in the set of body data—the second subset of body data representing movement of a set of anatomical features of the animal and/or facial expressions of the animal during capture of the video—and the lameness model, detect lameness of a lameness type (e.g., depression, anxiety, fear) corresponding to a particular mental health disorder associated with various postural behaviors and/or facial expressions of animals.

Additionally or alternatively, in another implementation, the computer system can predict lameness of a third subset of lameness types, in the set of lameness types, corresponding to pain experienced by the animal. For example, the computer system can: receive a video of an animal executing a series of target movements during a video capture session; extract a set of body data from the video; and detect lameness of a first lameness type—corresponding to pain experienced by the animal—based on a first subset of body data, in the set of body data, representing movement of a first subset of body features in a set of body features (e.g., limbs, joints, paws, snout, tail, facial features) of the animal.

The computer system can, therefore, leverage a single video of the animal performing a particular sequence of movements—such as including walking in various directions relative a device (e.g., the owner's mobile device) recording the video, various target poses (e.g., “sit”, “stand”, “lie down”), and/or transitions between poses (e.g., “sit to stand,” “stand to sit,” “sit to lie down, “lie down to sit”)—to predict presence and/or absence of lameness of a wide range of lameness types, including lameness associated with physical injury or ailment, disease, mental health disorders, and/or animal pain.

### 5.3.1 Lameness Score

In one implementation, the computer system can represent lameness of an animal—detected in a video of the dog—as a lameness score. In one example, the computer system can characterize lameness as a lameness score on a scale from one to five. In this example, the computer system can: predict a lameness score of one for an animal exhibiting minimal or no signs of lameness; predict a lameness score of three for an animal exhibiting a relatively moderate degree of lameness; and predict a lameness score of five for an animal exhibiting a relatively high degree of lameness. In another example, the computer system can represent lameness as a lameness score between zero percent and 100 percent. Alternatively, in yet another example, the computer system can characterize lameness on a binary scale, such as corresponding to presence or absence of lameness.

Additionally or alternatively, in one implementation, the computer system can characterize a lameness score for a particular lameness type. For example, the computer system can: predict a first lameness score associated with hip dysplasia based on a first set of body data extracted from a video captured during a video capture session with the dog; predict a second lameness score associated with elbow dysplasia based on a second set of body data extracted from the video; and predict a third lameness score associated with an ankle sprain based on a third set of body data extracted from the video. The computer system can therefore predict presence and/or absence of lameness for each lameness type, in a corpus of lameness types, thereby enabling comprehensive analysis of the dog's health.

In particular, in one example, the computer system can: receive a video of the animal executing a series of target movements during a video capture session; extract a set of body data from the video (e.g., as described above); for a first lameness type (e.g., a physical injury), predict a first lameness score—representing a likelihood of the animal exhibiting lameness of the first lameness type—based on a first subset of body data, in the set of body data, and the lameness model; and, for a second lameness type (e.g., a metal health disorder), predict a second lameness score—representing a likelihood of the animal exhibiting lameness of the second lameness type—based on a second subset of body data, in the set of body data, and the lameness model. In this example, in response to the first lameness score exceeding a threshold score, the computer system can predict lameness of the first lameness type for the animal. Furthermore, in response to the second lameness score falling below the threshold score, the computer system can predict absence of lameness of the second lameness type for the animal.

The computer system can thus generate a lameness score for each lameness type in a set of lameness types (e.g., defined for the animal). Based on the set of lameness scores, the computer system can predict presence and/or absence of each lameness type, in the set of lameness types, and thus generate a list of predicted lameness types exhibited by the animal in the video.

### 6. Variation: Body Condition Score

In one variation, the computer system can characterize a body condition score for the dog based on features extracted from a video of the dog captured by the user—and uploaded via the application—during a video capture session. Generally, in this variation, the computer system can extract a set of body data—representing size, shape, weight distribution, position, movement of the dog during the video capture session—from the video captured during the video capture session for the dog. The computer system can then leverage this set of body data to predict a body condition score—such as represented as a score between one and five—for the dog representative of the dog's health.

Additionally or alternatively, in this variation, the computer system can leverage one or more static images of the dog—such as in addition to or in replacement of video of the dog—to predict a body condition score for the dog. For example, the computer system can prompt the user to: capture a first image of the dog in a “sit” pose in a forward-facing position (e.g., facing the camera); capture a second image of the dog in the “sit” pose in a rear-facing position (e.g., facing away from the camera); capture a third image of the dog in the “sit” pose in a first side-facing position; and capture a fourth image of the dog in the “sit” pose in a second side-facing position opposite the first side-facing position. Additionally, the computer system can prompt the user to capture images of the dog in other poses (e.g., “stand,” “lie-down”) and/or orientations relative the camera. The computer system can then leverage body data extracted from these images to derive a body condition score for the dog.

The computer system can implement a body condition model—linking characteristics of dog shape, size, postures, movements, etc. to body condition scores of dogs—to predict a body condition score for the dog. In one implementation, the computer system can select a particular body condition model—from a corpus of existing body condition models—trained on images and/or video of dogs of an age, breed, size, etc. corresponding to characteristics of the dog (e.g., stored in the dog profile) and developed to detect lameness in dogs exhibiting characteristics similar to the dog. Similarly, the computer system can tune a generic body condition model based on characteristics stored in the dog profile. Alternatively, in another implementation, the computer system can implement a generic body condition model developed to predict body condition scores for dogs based on images or video of dogs. In each of these implementations, the computer system can then implement this body condition model to predict a body condition score for a dog based on features extracted from images or video of the dog.

### 7. Veterinarian Tool

Generally, the computer system can interface with a veterinarian portal executing on a computing device (e.g., a smartphone, a tablet, a laptop, a desktop computer) accessed by a veterinarian—or any other animal health professional—associated with the dog.

In one implementation, the veterinarian may: access an instance of the application on a computing device accessed by the veterinarian, such as by downloading an instance of a native application onto the computing device and/or navigating to an instance of a web application within a browser executing on the computing device; and access a veterinarian portal within the application. Similarly, the user (i.e., the dog owner) may: access an instance of the application on a computing device accessed by the user; and access an owner portal within the application. The computer system can then link the owner portal—accessed by the user—to the veterinarian portal accessed by the veterinarian, in order to enable communication and/or sharing of data (e.g., video) between the owner and veterinarian portals. In one example, a user may visit her veterinarian for a scheduled appointment with her animal. The veterinarian may then: access the owner portal on her computing device; and prompt the user to scan a QR code—rendered within the owner portal rendered on the veterinarian's computing device—with her mobile device to automatically download the application. The user may then scan the QR code—including a pointer to the veterinarian—with her mobile device to download the application and access the user portal automatically linked to the veterinarian portal via the pointer contained in the QR code. The computer system can then share video(s) of the dog—recorded by the user on her mobile device during one or more video capture sessions—with the veterinarian for further review.

### 7.1 Veterinarian Report

Blocks S140 and S142 of the method S100 recite, in response to detecting lameness of the first lameness type for the animal: generating a report indicating detection of lameness of the first lameness type; and transmitting the report to a second computing device accessed by an animal health professional affiliated with the animal.

In one implementation, in response to receiving a video recorded during a video capture session for a dog, the computer system can automatically generate a report summarizing insights derived from the video and related to dog health, such as corresponding to instances of lameness detected in the video.

Generally, in this implementation, the computer system can generate and transmit a report—indicating detection of lameness exhibited by the animal in the video of the animal executing the sequence of target movements—to a computing device accessed by the animal health professional prior to an appointment scheduled for the animal with the animal health professional.

In particular, the computer system can: receive a video recorded on a mobile device accessed by the user during a video capture session with the user's dog; and scan the video for quality issues (e.g., as described above) and selectively approve or reject the video accordingly. Then, in response to approving the video, the computer system can: extract a set of body data—including locations of the dog within a working field, relative positions of various body features (e.g., head, feet, knees, hips) of the dog's body during various poses, transitions, and/or movements, velocity of the dog's body, a weight distribution of the dog in a particular pose and/or during a particular transition or movement, etc.—from the video; implement the lameness model to detect instances of lameness—such as corresponding to a particular body feature and/or condition—exhibited by the dog in the video based on the set of body data; generate a report summarizing any instances of lameness detected in the video and/or including particular frames or clips of the video depicting these instances of lameness; and generate a prompt to review the report and/or the (original) video recorded during the video capture session—and transmit the prompt—including the report and the video—to the veterinarian via the veterinarian portal. The computer system can, therefore, immediately surface instances of lameness detected within the video—including clips and/or images supporting prediction of these instances of lameness—to the veterinarian for review, thereby enabling the veterinarian to more quickly confirm and/or disconfirm instances of lameness exhibited by the dog.

In one variation, the computer system can selectively transmit the report to the animal health professional in response to detecting lameness of a particular lameness type. In particular, in this variation, regardless of whether the animal is scheduled to visit the animal health professional within a particular time window, the computer system can automatically generate and transmit a report—indicating detection of lameness exhibited by the animal in the video of the animal executing the sequence of target movements—to the animal health professional for review. For example, in response to detecting lameness of a first lameness type associated with a high urgency level, the computer system can: generate a report indicating detection of lameness of the first lameness type for the animal; populate the report with a first prompt to review a segment of the video depicting lameness of the first lameness type exhibited by the animal; populate the report with a second prompt to contact the owner of the animal pending review of the video; and transmit the prompt to a computing device (e.g., via the veterinarian portal) accessed by the animal health professional. Additionally or alternatively, in this example, in response to detecting lameness of a second lameness type associated with a low urgency level, the computer system can: generate a report indicating detection of lameness of the second lameness type for the animal; populate the report with a prompt to review a segment of the video depicting lameness of the second lameness type exhibited by the animal; and queue the report for review by the animal health professional prior to a next scheduled appointment with the animal. Therefore, the computer system can: immediately notify the animal health professional regarding detection of high-urgency (or high-risk) lameness types exhibited by an animal, thereby enabling earlier detection of lameness and/or earlier implementation of a corresponding treatment pathway; and minimize resources dedicated to review of low-urgency (or low-risk) lameness types outside of scheduled appointments or defined pre-appointment review periods.

In one implementation, as described above, the computer system can interface with a patient management system (e.g., a cloud platform) employed by an animal health professional and/or animal health network. In this implementation, the computer system can transmit the report to the animal health professional directly via the patient management system, thereby enabling the animal health professional to access this report in addition to all other health records, notes, etc. associated with this animal health professional (or network including the animal health professional) in a singular location. Furthermore, in this implementation, the computer system can: receive a request for execution of a video capture session—for a particular animal affiliated with a particular owner—from the animal health professional via the patient management system.

### 7.1.1 Treatment Pathway

In one variation, the computer system can populate the report with a suggested treatment pathway based on a particular type of lameness exhibited by the dog. In particular, in this variation, the computer system can: selectively identify a particular treatment pathway—tailored to a type of lameness (e.g., acute injury, chronic injury, disease, condition) detected in a video of the dog—configured to mitigate and/or alleviate symptoms associated with the type of lameness exhibited by the dog; populate the report with a prompt to review the particular treatment pathway; and transmit the report to the veterinarian via the veterinarian portal.

Additionally or alternatively, in this variation, the computer system can leverage known characteristics of the animal—in combination with the lameness type—to suggest a particular treatment pathway. For example, the computer system can: receive a video of an animal executing a series of target movements during a video capture session; extract a set of body data from the video; and, based on a first set of body data and the lameness model, detect lameness of a first lameness type exhibited by the animal in the video; access an animal profile, in a population of animal profiles, generated for the animal; extract a set of animal characteristics—such as including a breed, a size, a set of primary coat colors of the dog's coat, etc.—defined for the animal in the animal profile; and, based on the first lameness type and the set of animal characteristic, select a first treatment pathway predicted to mitigate lameness of the first lameness type in animals exhibiting characteristics approximating the set of characteristics of the animal. In this example, the computer system can then: generate a report indicating detection of lameness of the first lameness type for the animal; append the report with a suggestion to implement the first treatment pathway; and transmit the report to the animal health professional (e.g., via the veterinarian portal).

### 7.2 Veterinarian: Pre-Appointment Video Session

In one variation, the computer system can prompt the user to capture a video of her dog prior to an upcoming appointment with the dog's veterinarian. The computer system can then: receive the video from the user (e.g., via the native or web application); derive insights related to the dog's health—such as related to lameness, body condition score, dermatology, neurological health, etc.—based on features extracted from the video; summarize these insights in a report for the veterinarian; and transmit the report and corresponding video to the veterinarian—via the veterinarian portal—for review prior to the upcoming appointment with the dog.

In particular, in this variation, the computer system can: at a first time, access a schedule defined for the animal health professional (e.g., a veterinarian); and identify an appointment for the animal—such as scheduled by the animal owner—and specified by the schedule, the appointment scheduled at a second time succeeding the first time and falling within a threshold duration (e.g., 24 hours, 72 hours, one week, one month) of the first time. Then, in response to identifying the appointment scheduled for the animal at the second time, the computer system can: generate an owner prompt to capture a video of the animal executing the series of target movements (e.g., according to a session protocol); and transmit the owner prompt to the owner at a first computing device associated with the owner, such as via an instance of an owner portal executing on the first computing device. The computer system can then receive this video—captured at the computing device of the owner—at a third time succeeding the second time, such as via the instance of the owner portal.

For example, the computer system can: access an appointment schedule defined for a veterinarian; identify an appointment for a particular dog scheduled at a time within a particular time period, such as in the next several days or in the next week; generate an owner prompt to execute a video capture session with her dog and upload a video of her dog—executing a capture session protocol during the video capture session accordingly—via the application (e.g., a native or web application); transmit the owner prompt to the dog's owner via an instance of the owner portal affiliated with the user; scan the video and implement a lameness model to detect instances of lameness exhibited by the dog in the video; generate a report (e.g., as described above) detailing any instances of lameness detected in the video and/or any other key information extracted from the video; generate a veterinarian prompt to review the report and/or the video prior to the appointment with the dog and the dog's owner; and transmit the veterinarian prompt to the veterinarian for review of the report and/or the video prior to the appointment.

Additionally, in the preceding example, in response to detecting one or more instances of lameness—such as corresponding to injury or pain exhibited by the animal—the computer system can: generate an owner report indicating detection of lameness of a particular type and/or magnitude exhibited by her dog and including a set of instructions for caring for her dog prior to the appointment. In this example, the computer system can provide these instructions to the dog's owner—prior to receiving a confirmed diagnosis of lameness from the veterinarian—in order to avoid worsening of lameness and/or minimize pain experienced by the dog; and transmit the owner report to the dog's owner.

### 7.3 Veterinarian: Periodic Check-Ins

Additionally or alternatively, in another implementation, the computer system can prompt the user to periodically execute a video capture session with the dog, such as weekly, monthly, etc. The computer system can then execute Blocks of the method S100, as described above, to scan videos captured during these video capture sessions; detect instances of lameness and/or predict body condition scores for the dog based on body data extracted from these videos; and generate reports for the user's veterinarian to review and/or for the user to review.

In one implementation, the computer system can: store a timeseries of reports—each linked to a particular video, in a series of videos, recorded during a video capture session with the dog—generated for a timeseries of video capture sessions with the dog in the dog profile; and prompt the veterinarian—via the veterinarian portal—to review the timeseries of reports at a fixed frequency and/or prior to an appointment with the animal. The computer system can therefore maximize an amount of data collected for the dog while minimizing an amount of time spent by the veterinarian reviewing this data.

Additionally or alternatively, in one implementation, the computer system can alert the veterinarian to review a particular report—linked to a particular video captured during a video capture session with the dog—responsive to detecting an instance of lameness exhibited by the dog. For example, at a first time, the computer system can: prompt the user to capture a first video of her dog during a first video capture session; implement the lameness model to detect instances of lameness exhibited by the dog in the first video; and, in response to detecting absence of lameness in the first video, generate a first report—including a link to the first video—indicating absence of lameness in the first video and store the first report in the dog profile (e.g., for selective review by the veterinarian). Later, at a second time, the computer system can: prompt the user to capture a second video of her dog during a second video capture session; and implement the lameness model to detect instances of lameness exhibited by the dog in the second video. Then, in response to detecting a first instance of lameness corresponding to hip dysplasia, the computer system can: generate a second report—including a link to the second video—indicating detection of the first instance of lameness for the dog; generate a notification including the second report and a prompt to review the second report and/or to contact the user to schedule an appointment with her dog; and transmit the notification to the veterinarian via the veterinarian portal for immediate review. The computer system can therefore monitor the dog's health—such as characterized by lameness and/or body condition score—over time and selectively alert the veterinarian responsive to changes in the dog's health and/or detection of health-related issues associated with worsening of the dog's health.

### 7.4 Veterinarian Feedback+Treatment Pathway Effectiveness

In one variation, the computer system can prompt the veterinarian to selectively confirm or disconfirm an instance of lameness—such as of a particular type—detected in a video of the dog. The computer system can then update the lameness model based on whether the veterinarian confirms or disconfirm this instance of lameness. For example, the computer system can: generate a first report detailing an instance of lameness detected in a video of a dog; generate a first prompt to review the first report and/or the video prior to an appointment with the dog and the dog's owner; and transmit the veterinarian prompt to the veterinarian for review of the report and/or the video prior to the appointment. Then, in response to completion of the appointment with the dog and the dog's owner, the computer system can: generate a second prompt to confirm or reject the instance of lameness detected in the video, such as based on further examination of the dog by the veterinarian during the appointment; and, in response to the veterinarian confirming the instance of lameness, update the lameness model to reflect correct detection of the instance of lameness. Alternatively, in response to the veterinarian rejecting the instance of lameness, the computer system can rectify the model based on absence of lameness in the first video and corresponding frames of the first video.

Additionally or alternatively, in one variation, the computer system can track effectiveness of a treatment pathway selected by the physician for the dog—responsive to detection of lameness of a particular type for the dog—based on changes in body data derived from video and/or images of the dog over time. For example, the computer system can: detect a first instance of lameness of a first type for a dog based on a first video captured during a first video capture session with the dog; generate a report indicating detection of the first instance of lameness of the first type; transmit the report to the dog's veterinarian for review prior to an appointment with the dog. After further examination of the dog during the appointment, the veterinarian may: confirm presence of lameness of the first type for the dog; and select a particular treatment pathway (e.g., medication, exercise) configured to treat lameness of the first type in dogs. Later, based on a second video of the dog captured during a second video capture session—succeeding the appointment with the veterinarian—the computer system can: detect a second instance of lameness of the first type for the dog; characterize a difference—such as a difference in magnitude, symptoms present, and/or detectability—between the first instance of lameness of the first type and the second instance of lameness; and characterize effectiveness of the particular treatment pathway based on the difference. The computer system can then: generate a second report indicating effectiveness of the first treatment pathway and including a prompt to review changes between the first and second instance of lameness for the dog between the first and second video capture sessions; and transmit the report to the veterinarian for review. In this variation, the computer system can thus prompt the owner to capture additional video recordings of the animal to enable tracking of effectiveness of a particular treatment pathway over time.

### 8. Variation: Pet Owner Tool

Additionally or alternatively, in one variation, the computer system can provide feedback related to dog health and derived from video of the dog—such as corresponding to detection of lameness, body condition scores, etc.—directly to the user (i.e., the pet owner).

In this variation, Blocks S150 and S152 of the method S100 recite, in response to detecting lameness of the first lameness type for the animal: generating a second report indicating detection of lameness of the first lameness type and including a prompt to review the second report with the animal health professional; and transmitting the second report to the owner at the first computing device.

In particular, in this variation, the user may initiate a video capture session with her dog—such as unprompted and/or responsive to receiving a prompt from the computer system via the application—and capture a video of the dog accordingly. The computer system can then: receive the video via the application; scan the video for quality issues (e.g., as described above) and selectively approve or reject the video accordingly; extract a set of body data from the video; implement the lameness model to detect instances of lameness exhibited by the dog in the video based on the set of body data; generate a report summarizing any instances of lameness detected in the video and/or including particular frames or clips of the video depicting these instances of lameness; and transmit the report to the user.

Furthermore, the computer system can selectively provide suggestions to the user in response to detecting an instance of lameness for the dog. For example, in response to detecting an instance of lameness in a video of the dog, the computer system can: generate a report indicating detection of the instance of lameness; select a treatment pathway, from a set of treatment pathways, associated with treatment of the instance of lameness; generate a prompt to schedule an appointment with the dog's veterinarian for further examination of the instance of lameness and/or for discussion of the suggested treatment pathway with the veterinarian; append the report with the prompt; and transmit the report to the user via the owner portal.

### 9. Variation: Ambient Video Collection

In one variation, the computer system can leverage videos of the dog collected by ambient sensors installed at (e.g., inside and/or outside) a home of the dog owner—and/or other locations frequented by the dog (e.g., a boarding facility, the dog owner's vehicle)—to periodically derive insights related to health of the dog based on data extracted from these videos. Generally, in this variation, the computer system can: discard segments of a video feed—captured by an ambient optical sensor installed in the user's home—in which the dog is absent from the video feed; and store segments of the video feed in which the dog is present and/or detected in the video feed.

In one implementation, the computer system can access video captured by an optical sensor (e.g., a doorbell camera) integrated within a doorbell (e.g., an internet-connected, wireless video doorbell) installed at the dog's home. In particular, in this implementation, the computer system can: access a video feed captured by an optical sensor integrated into a doorbell, such as installed outside the user's home; and implement the animal model to detect presence and/or absence of the dog within the video feed. Then, in response to detecting presence of the dog in a particular segment of the video feed, the computer system can: extract a set of body data—such as including a sequence of locations of the dog within a working field, a sequence of relative positions of various body features (e.g., head, feet, knees, hips) of the dog's body, velocities of the dog's body during particular transitions or movements, a weight distribution of the dog in a particular pose and/or during a particular transition or movement, etc.—from the segment of the video feed; and implement the lameness model to detect instances of lameness for the dog within the segment of the video feed. In a similar implementation, the computer system can access video captured by an optical sensor installed in a video monitor (e.g., a security system, a dog monitor)—or any other type of home device—installed in the user's home.

In one variation, the computer system can prompt the user to execute a video capture session with the dog in response to detecting an instance of lameness for the dog in an ambient video captured by an ambient optical sensor installed in and/or about the user's home. For example, in this variation, the computer system can: access a video feed captured by an ambient optical sensor integrated within a home device (e.g., a doorbell, a security system, a monitor) installed in the user's home; implement the animal model to detect presence and/or absence of the dog within the video feed; in response to detecting presence of the dog in a particular segment of the video feed, extract a set of body data from the segment of the video feed; and implement the lameness model to detect instances of lameness for the dog within the segment of the video feed. Then, in response to detecting a first instance of lameness for the dog, the computer system can: generate a notification indicating detection of the first instance of lameness and including a prompt to execute a video capture session—such as according to a video session protocol—with the dog; and transmit the notification to the user (e.g., via text message, via push notification). The computer system can thus selectively notify the user to execute a video capture session—from which the computer system can derive higher resolution data for the dog—responsive to initial detection of lameness for the dog in video captured by an ambient optical sensor installed in the user's home.

### 9.1 Ambient Sensor Data: Video+Audio Data Collection

In one variation, the computer system can leverage recordings (e.g., video recordings, audio recordings) of the dog collected by ambient sensors installed at and/or within the home of the owner of the dog—and/or other locations frequented by the dog—to periodically derive insights related to health of the dog based on data extracted from these recordings. For example, the computer system can access video recordings of the dog captured by an optical sensor integrated within a doorbell, integrated within a vehicle of the dog owner, within a home security system, etc.

In particular, in this variation, the computer system can: receive a recording of the animal captured by an ambient sensor installed at and/or within a home of the owner of the animal; in response to detecting presence of the animal within the recording (e.g., via implementation of the animal model), extract a set of animal lameness data—such as including video and/or audio data—from the recording; and, based on the set of animal lameness data and the lameness model, predict presence and/or absence of lameness of a set of lameness types for the animal at a first confidence level for the animal.

For example, as described above, the computer system can: receive a video recording captured by an optical sensor integrated within a doorbell system installed at the home of the owner of the animal; extract a set of body data—representing movement of the set of body features of the animal depicted in the video recording—from the video recording; and predict presence and/or absence of lameness of a set of lameness types for the animal based on the set of body data and the lameness model. Additionally or alternatively, in another example, the computer system can: receive an audio recording captured by a microphone integrated within a smart home assistant system installed at the home of the owner of the animal; extract a set of audio data—representing animal vocalizations output by the animal—from the audio recording; and predict presence and/or absence of lameness of a set of lameness types for the animal based on the set of audio data and the lameness model.

Additionally, in this variation, the computer system can similarly prompt the user to execute a video capture session with the dog in response to detecting an instance of lameness for the dog in an ambient audio and/or video recording captured by an ambient sensor installed in and/or about the user's home. For example, the computer system can: receive a recording of the animal captured by an ambient sensor (e.g., an audio sensor, an optical sensor) installed at the home of the owner; extract a set of animal lameness data from the recording; and, based on the set of animal lameness data and the lameness model, predict lameness of a first lameness type, in a set of lameness types, at a first confidence level—exceeding a threshold confidence—for the animal. Then, in response to predicting lameness of the first lameness type at the first confidence level for the animal, the computer system can: generate a prompt to execute a first video capture session with the animal (e.g., according to a session protocol); and transmit the prompt to the owner (e.g., via the owner portal).

### 10. Variation: Remote Scanning

In one variation, the computer system can interface with an animal scanning device—including a set of optical sensors configured to capture video and/or images of dogs—installed at a location visited by pet owners and corresponding dogs. For example, the computer system can interface with an animal scanning device installed at a particular location, such as a pet store, a veterinarian clinic, an animal boarding facility, etc. In this example, the user may bring her dog to the particular location to execute a video capture session for her dog via the animal scanning device. Rather than record a video of the dog on the user's mobile device and/or uploading the video via the application (e.g., as described above), the animal scanning device can then: initiate a video capture session with the dog; and capture a video of the dog executing a video session protocol during the video capture session. The computer system can then: scan the video and implement a lameness model to detect instances of lameness exhibited by the dog in the video; generate a report detailing any instances of lameness detected in the video and/or any other key information extracted from the video; and transmit the report to the user (e.g., via test message, via the application). Additionally or alternatively, in this example, the computer system can: implement a body condition model to derive a body condition score for the animal based on features extracted from the video; and populate the report with the body condition score derived for the animal.

Furthermore, in the preceding example, the computer system can populate the report with a suggested treatment pathway based on insights derived from the video captured during the video capture session. For example, in response to detecting an instance of lameness related to an acute injury, the computer system can populate the report with a suggested treatment pathway of rest for the dog and/or visiting the dog's veterinarian. In another example, in response to detecting an instance of lameness related to a disease, the computer system can populate the report with a suggested treatment pathway of scheduling an appointment with the dog's veterinarian for further evaluation and/or medication recommendations. In yet another example, in response to detecting an instance of lameness related to pain associated with overgrown nails, the computer system can populate the report with a suggested treatment pathway of clipping the dog's nails and/or scheduling an appointment with a dog groomer.

In one implementation, the user may initially download and/or navigate to the application (e.g., a native or web application) and generate a dog profile for her dog as described above. Then, when the user visits the particular location—for execution of a video capture session with her dog via the animal scanning device—the computer system can identify the user's dog and access the corresponding dog profile. For example, when the user visits the particular location—for execution of a video capture session with her dog via the animal scanning device—the computer system and/or animal scanning device can prompt the user to scan an RFID tag located on a collar of the dog. In particular, in one example, the computer system can: prompt a first user to scan an RFID tag located on a collar of a first dog associated with the first user; and, in response to the RFID tag corresponding to the first dog, the computer system can access a first dog profile corresponding to the first dog. Later, the computer system can: prompt a second user to scan an RFID tag located on a collar of a second dog associated with the second user; and, in response to the RFID tag corresponding to the second dog, the computer system can access a second dog profile corresponding to the second dog.

In another example, the computer system and/or animal scanning device can prompt the user to scan a unique QR code—linked to the dog profile generated for this user's dog—rendered within the application accessed on the user's mobile device. In particular, in one example, the computer system can: prompt a first user to scan a first QR code rendered with a first instance of the application accessed by the first user; and automatically access a first dog profile—corresponding to a first dog associated with the first user—linked to the first QR code. Later, the computer system can: prompt a second user to scan a second QR code rendered with a second instance of the application accessed by the second user; and automatically access a second dog profile—corresponding to a second dog associated with the second user—linked to the second QR code.

The computer system and/or animal scanning device can therefore distinguish between multiple dogs and store data—including video content and/or insights related to lameness, body condition score, etc.—derived for a particular dog in a corresponding dog profile.

The computer systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
receiving a video of an animal executing a series of target movements during a first video capture session, the video captured by a first computing device accessed by an owner of the animal;
extracting a set of body data from the video, the set of body data representing movement of a set of body features of the animal depicted in the video;
accessing a lameness model linking body data extracted from videos of animals to lameness of a set of lameness types in animals;
based on the set of body data and the lameness model, predicting lameness of a first lameness type exhibited by the animal in a first segment of the video; and
in response to detecting lameness of the first lameness type for the animal:
generating a report indicating detection of lameness of the first lameness type;
populating the report with the segment of the video corresponding to lameness of the first lameness type; and
transmitting the report to a second computing device accessed by an animal health professional affiliated with the animal.

2. The method of claim 1:
further comprising:
at a first time, accessing a schedule defined for the animal health professional;
identifying a first appointment for the animal and defined by the schedule, the first appointment scheduled at a second time succeeding the first time and falling within a threshold duration of the first time; and
in response to identifying the first appointment scheduled for the animal at the second time:
generating a first owner prompt to capture the video of the animal executing the series of target movements; and
transmitting the first owner prompt to the owner at the first computing device; and
wherein receiving the video captured by the first computing device comprises, at a third time succeeding the first time and preceding the second time, receiving the video captured by the first computing device.

3. The method of claim 1, further comprising, in response to detecting lameness of the first lameness type for the animal:
generating a second report indicating detection of lameness of the first lameness type and comprising a prompt to review the second report with the animal health professional; and
transmitting the second report to the owner at the first computing device.

4. The method of claim 1, wherein detecting lameness of the first lameness type based on the set of body data comprises detecting lameness of the first lameness type based on a first subset of body data, in the set of body data, representing movement of a set of limbs, in the set of body features, of the animal, the first lameness type corresponding to a physical injury within a first limb in the set of limbs.

5. The method of claim 1, wherein detecting lameness of the first lameness type based on the set of body data comprises detecting lameness of the first lameness type based on a first subset of body data, in the set of body data, representing movement of a first subset of body features, in the set of body features, of the animal, the first lameness type corresponding to pain experienced by the animal.

6. The method of claim 1, wherein receiving the video of the animal executing the series of target movements comprises receiving the video of the animal executing the series of target movements comprising:
walking in a first direction along a pathway within a field of view of an optical sensor integrated within the first computing device;
walking in a second direction along the pathway, the second direction opposite the first direction; and
transitioning from a sit position to a stand position.

7. The method of claim 1:
further comprising, in response to receiving the video from the first computing device:
characterizing a quality of the video; and
in response to the quality exceeding a threshold quality, approving the video for analysis; and
wherein extracting the set of body data from the video comprises, in response to approving the video for analysis, extracting the set of body data from the video.

8. The method of claim 7, further comprising, in response to the quality falling below the threshold quality:
generating a first owner prompt to record a second video of the animal executing a subseries of target movements, in the series of target movements, during a second video capture session; and
transmitting the first owner prompt to the owner at the first computing device.

9. The method of claim 1, wherein extracting the set of body data representing movement of the set of body features of the animal comprises extracting the set of body data representing:
movement of a set of anatomical features of the animal during execution of the series of target movements; and
facial expressions of the animal during execution of the series of target movements.

10. The method of claim 1:
further comprising:
for the first lameness type, predicting a first lameness score based on a first subset of body data, in the set of body data, and the lameness model, the first lameness score representing a likelihood of the animal exhibiting lameness of the first lameness type; and
for a second lameness type, in the set of lameness types, predicting a second lameness score based on a second subset of body data, in the set of body data, and the lameness model, the second lameness score representing a likelihood of the animal exhibiting lameness of the second lameness type;
wherein detecting lameness of the first lameness type comprises, in response to the first lameness score exceeding a threshold score, predicting lameness of the first lameness type; and
further comprising, in response to the second lameness score falling below the threshold score, predicting absence of lameness of the second lameness type for the animal.

11. The method of claim 1:
wherein predicting lameness of the first lameness type based on the set of body data and the lameness model comprises predicting lameness of the first lameness type based on a first subset of body data, in the set of body data, and the lameness model, the first subset of body data associated with the first lameness type comprising a physical injury; and
further comprising predicting lameness of a second lameness type based on a second subset of body data, in the set of body data, and the lameness model, the second subset of body data associated with the second lameness type comprising a mental health disorder.

12. The method of claim 1:
further comprising:
annotating the segment of the video with a set of annotations configured to highlight lameness of the first lameness type exhibited by the animal; and
appending the report with a prompt to review the segment of the video and further investigate lameness of the first lameness type in the animal; and
wherein transmitting the report to the second computing device accessed by the animal health professional comprises transmitting the report to the second computing device accessed by the animal health professional prior to an appointment scheduled for the animal with the animal health professional.

13. The method of claim 12, further comprising:
accessing an animal profile, in a population of animal profiles, generated for the animal;
extracting a set of animal characteristics defined for the animal in the animal profile;
selecting a first treatment pathway, in a set of treatment pathways, based on the first lameness type and the set of animal characteristics; and
appending the report with a suggestion to implement the first treatment pathway to mitigate lameness of the first lameness type exhibited by the animal.

14. The method of claim 1:
wherein receiving the video captured by the first computing device comprises receiving the video captured by the first computing device at a first time; and
further comprising:
at a second time preceding the first time, receiving a recording of the animal captured by an ambient sensor installed at a home of the owner;
extracting a set of animal lameness data from the recording;
based on the set of animal lameness data and the lameness model, predicting lameness of the first lameness type at a first confidence level for the animal, the first confidence level exceeding a threshold confidence; and
in response to predicting lameness of the first lameness type at the first confidence level for the animal:
generating a prompt to execute the first video capture session with the animal; and
transmitting the prompt to the owner.

15. The method of claim 14:
wherein receiving the recording captured by the ambient sensor installed at the home of the owner comprises receiving a video recording captured by an optical sensor integrated within a doorbell system installed at the home of the owner; and
wherein extracting the set of animal lameness data from the recording comprises extracting a second set of body data from the video recording, the second set of body data representing movement of the set of body features of the animal depicted in the video recording.

16. The method of claim 14:
wherein receiving the recording captured by the ambient sensor, installed at the home of the owner, comprises receiving an audio recording captured by a microphone integrated within a smart home assistant system installed at the home of the owner; and
wherein extracting the set of animal lameness data from the recording comprises extracting a set of audio data from the audio recording, the set of audio data representing animal vocalizations output by the animal.

17. A method comprising:
at a first time, accessing a schedule defined for an animal health professional;
identifying a first appointment for the animal and defined by the schedule, the first appointment scheduled at a second time succeeding the first time and falling within a threshold duration of the first time;
in response to identifying the first appointment:
generating a first owner prompt to capture a video of the animal executing a series of target movements during a video capture session; and
transmitting the first owner prompt to an owner of the animal via an instance of an owner portal executing on a first computing device accessed by the owner;
in response to receiving the video of the animal the instance of the owner portal:
extracting a set of body data from the video, the set of body data representing movement of a set of body features of the animal depicted in the video;
accessing a lameness model linking body data extracted from videos of animals to lameness of a set of lameness types in animals; and
based on the set of body data and the lameness model, detecting lameness of a first lameness type exhibited by the animal in a first segment of the video; and
in response to detecting lameness of the first lameness type for the animal:
generating a report indicating detection of lameness of the first lameness type;
extracting a portion of the video depicting lameness of the first lameness type exhibited by the animal;
populating the report with a prompt to review the portion of the video; and
at a third time succeeding the first time and preceding the second time, transmitting the report to an animal health professional for review via an instance of a veterinarian portal associated with the animal health professional.

18. A method comprising:
receiving a video of an animal executing a series of target movements during a first video capture session;
extracting a set of body data from the video, the set of body data representing characteristics of a set of body features of the animal depicted in the video;
accessing a lameness model linking body data extracted from videos of animals to lameness of a set of lameness types in animals;
based on the set of body data and the lameness model, detecting lameness of a first lameness type exhibited by the animal in a first segment of the video; and
in response to detecting lameness of the first lameness type for the animal:
generating a report indicating detection of lameness of the first lameness type;
populating the report with the segment of the video corresponding to lameness of the first lameness type; and
transmitting the report to a first computing device accessed by a user affiliated with the animal.

19. The method of claim 18:
wherein receiving the video of the animal comprises receiving the video of the animal from a second computing device accessed by an owner of the animal; and
wherein transmitting the report to the first computing device accessed by the user affiliated with the animal comprises transmitting the report to the first computing device accessed by an animal health professional affiliated with the animal.

20. The method of claim 18:
further comprising populating the report with a prompt to review the report with an animal health professional; and
wherein transmitting the report to the first computing device, accessed by the user affiliated with the animal, comprises transmitting the report to the first computing device accessed by an owner of the animal.
